(19) United States
(12) Patent Application Publication
(10) Pub. No.: US 2024/0378890 A1
(43) Pub. Date: Nov. 14, 2024

(54) In-Bed Pose and Posture Tracking System

(71) Applicant: Northeastern University, Boston, MA (US)

(72) Inventors: Sarah OSTADABBAS, Watertown, MA (US); Shuangjun LIU, Boston, MA (US)

(21) Appl. No.: 18/696,091

(22) PCT Filed: Oct. 31, 2022
(86) PCT No.: PCT/US2022/048375

§ 371 Date: Mar. 27, 2024

Related U.S. Application Data
(60) Provisional application No. 63/273,486, filed on Oct. 29, 2021.

(51) Int. Cl. G06V 20/50; G06V 10/50; G06V 10/764; G06V 10/94; G06V 40/10

(52) U.S. Cl. CPC G06V 20/50; G06V 10/50; G06V 10/764; G06V 10/95; G06V 40/10; G06V 2201/03

(57) ABSTRACT

Systems and methods are provided for in-bed pose and posture determination and tracking for a human subject including an imaging device, the imaging device positioned proximate to a bed and oriented to capture images of the subject lying in the bed, and a processing unit operative to receive the captured images, the captured images including a plurality of image frames, the processing unit including a pose estimation module trained with a dataset of lying poses and operative to estimate poses of the subject lying in the bed based the image frames, and a posture classification module trained with the dataset of lying poses and operative to classify positions of the subject lying in the bed based on the image frames, the processing unit operative to determine a pose and posture of the subject lying in the bed.

### STATEMENT REGARDING FEDERALLY SPONSORED RESEARCH OR DEVELOPMENT

This application claims benefit under 35 U.S.C. § 119 (e) of U.S. Provisional Application No. 63/273,486, filed on 29 Oct. 2021, entitled “In-Bed Pose and Posture Tracking System,” the entirety of which is incorporated by reference herein.

### STATEMENT REGARDING FEDERALLY SPONSORED RESEARCH OR DEVELOPMENT

This invention was made with government support under Grant Number 1755695 awarded by NSF National Science Foundation. The government has certain rights in the invention.

### BACKGROUND

Every person spends around one-third of their life in bed. For an infant or a young toddler this percentage can be much higher, and for bed-bound patients it can go up to 100% of their time. The quality of sleep postures has been consistently associated with many different health disorders and post-op complications. Past research has shown that sleeping in the supine position is detrimental to patients with sleep apnea and chronic respiratory problems, which could also put the fetus in danger during late pregnancy. The effects are not limited to pre-existing conditions; sleep posture has also been found to be a main contributing factor in developing pressure ulcers in post-surgical and elderly patients.

There have been many efforts over the last decade in the field of in-bed behavior tracking, with solutions ranging from using wearable sensors to external visual tracking. Some of these efforts are:

Wearable Devices: One approach involves using a textile-based intelligent wearable vest with a multi-channel inertial sensing system using five small sized accelerometers for multi-posture monitoring, gait analysis, and body balancing measurements. Since accelerometers are relatively ubiquitous and inexpensive, this is a viable approach. However, in such systems the subject must wear an external device for an extended period of time, potentially interfering with sleep patterns or, worse, being rejected by the subject as too cumbersome.

Pressure Mapping: Prior research has shown that data from pressure sensors can be considered as a gray-scale pressure image and trained on ensemble methods such as principal component analysis (PCA), k-nearest neighbor (kNN) or even using neural networks such as autoencoder methods. The downsides to using pressure sensors are portability, cost, and maintenance. That is, pressure sensors and pressure mats are not easy to move around or properly disinfect and require calibration for each new subject due to sensor drift.

Vision-based Methods: The topic of non-contact human pose and posture estimation has received a lot of attention in the last few years in the computer vision community thanks to the introduction of deep learning and its power in inference and modeling. However, the state-of-the-art vision-based algorithms used in this field face many challenges when targeting in-bed human behavior monitoring. Such challenges include significant illumination changes (e.g., full darkness at night), heavy occlusion (e.g., subjects covered by sheets, blankets, quilts, and/or other bedding/bedcoverings), as well as privacy concerns.

Such state-of-the-art systems are based on RGB images of the patient from a top-down position, accompanied by machine leaning algorithms to extract pose/posture related features. However, as noted above, image quality in such systems suffers when subjected to illumination changes, particularly in low-light nighttime environments (i.e., the conditions under which subjects are most likely to be in bed). Image quality also suffers in the presence of sheets, blankets, quilts, and/or other bedding/bedcoverings, often leaving the subject a no-win choice between sleeping uncomfortably without bedding or defeating the pose and posture estimation functionalities of the system.

With regard to privacy, the collection and recordation of images containing visually identifiable subjects in the privacy of their own home, particularly in bed, discourages adoption of such systems and, at a minimum, mitigates subject permission for large-scale data collection and use, which is necessary for deep model training. Moreover, the state-of-the-art deep learning-based pose/posture models are resource-intensive and therefore typically must be implemented in desktop or other settings with access to high performing GPUs.

By contrast, edge devices and other low-resource devices are unable to perform the same functions for real-world applications in the comfort of a person's home. Other modalities have been introduced in an attempt to circumvent these issues for in-bed human monitoring tasks, yet these modalities are achieved by providing only limited functionalities such as detection of a subject getting on/off the bed or narrow focus on only a small portion of the subject's body parts such as, for example, only head and torso.

### SUMMARY

As discussed above, in-bed behavior monitoring is commonly needed for bed-bound patients and has long been confined to wearable devices or expensive pressure mapping systems. Meanwhile, vision-based human pose and posture tracking, while experiencing a lot of attention/success in the computer vision field, has been hindered in terms of usability for in-bed cases, due to privacy concerns surrounding this topic. Moreover, the inference models for mainstream pose and posture estimation often require excessive computing resources, impeding their implementation on edge devices.

The technology described herein introduces a privacy-preserving in-bed pose and posture tracking system running entirely on an edge device and/or a cloud system with added functionality to detect stable motion as well as setting user-specific alerts for given poses and/or postures. This can be achieved by implementing vision-based feature extraction and image processing techniques such as histogram of oriented gradients (HoGs) in connection with imaging modalities such as long-wave infrared (LWIR), which are privacy-preserving and can work in complete darkness, preserving human pose information even when the person is fully covered under bedding/bedcoverings (e.g., sheets, blankets, quilts, and/or other bedding/bedcoverings). Advantageously, such modalities generally do not require the use of any additional sensors/equipment, reducing setup cost and maintenance time. In addition, the non-contact nature of these imaging modalities enhance the unobtrusiveness of the technology.

As described below, the estimation accuracy of the system was evaluated on a series of retrospective infrared (LWIR) images as well as samples from a real-world test environment. The test results reached over 93.6% estimation accuracy for in-bed poses and achieved over 95.9% accuracy in estimating three in-bed posture categories.

The technology described herein provides an in-bed pose and posture tracking system that is capable of performing pose and posture estimation on any mainstream cellphone as an edge device in a privacy preserving setting.

In one aspect, a system for in-bed pose and posture determination and tracking for a human subject is provided. The system includes an imaging device comprising one or more of a depth sensor or a long wavelength infrared camera, the imaging device positioned proximate to a bed and oriented to capture images of the subject lying in the bed. The system also includes a processing unit in communication with the imaging device and operative to receive the captured images of the subject lying in the bed, the captured images including a plurality of image frames, the processing unit comprising one or more processors and memory. The processing unit includes a pose estimation module trained with a dataset of lying poses and operative to estimate poses of the subject lying in the bed based on one or more of the image frames. The processing unit also includes a posture classification module trained with the dataset of lying poses and operative to classify positions of the subject lying in the bed based on one or more of the image frames. The processing unit is operative to determine a pose and posture of the subject lying in the bed.

In some embodiments, the imaging device is capable of imaging body pose and posture of the subject through bedding covering the subject. In some embodiments, the processing unit is integrated with the imaging device. In some embodiments, the processing unit is located remotely from the imaging device and in electronic communication with the imaging device. In some embodiments, the processing unit is located in a cloud-based server. In some embodiments, the pose estimation module includes a stacked hourglass model trained with the dataset of lying poses. In some embodiments, the posture classification module includes an autoencoder. In some embodiments, the autoencoder is a histogram of oriented gradients (HoG)-autoencoder. In some embodiments, the processing unit further comprises a preprocessor configured to compute HoG features of each of the one or more images received by the processing unit to form a HoG feature vector corresponding to each respective one of the one or more images. In some embodiments, the HoG-autoencoder includes an encoder configured to receive at least one of the HoG feature vectors formed by the preprocessor. In some embodiments, the HoG-autoencoder also includes an encoder configured to convert each respective HoG feature vector to a latent vector comprising a low-dimensional representation of the corresponding HoG feature vector. In some embodiments, the HoG-autoencoder also includes an output layer including a decoder trained to remap the latent vector to a HoG feature vector. In some embodiments, the HoG-autoencoder also includes a linear classification layer configured to determine a posture class probability for the corresponding image.

In some embodiments, the system also includes an edge device in communication with the processing unit and operative to receive images from the processing unit. In some embodiments, the imaging device and the processing unit are integrated within the edge device. In some embodiments, the system also includes an edge device in communication with the processing unit and operative to request notification of a detection of a type or duration of pose or posture by the processing unit. In some embodiments, the system also includes a motion detection module operative to determine if a same posture is returned from the posture classification module after a predetermined number of consecutive image frames. In some embodiments, the posture estimation model includes a single linear layer operative to classify positions of the subject lying in the bed based on pose estimation keypoints generated by the pose estimation module.

In another aspect, a method for in-bed pose and posture determination and tracking is provided. The method for in-bed pose and posture determination and tracking includes providing a system for in-bed pose and posture determination and tracking for a human subject. The system includes an imaging device comprising one or more of a depth sensor or a long wavelength infrared camera, the imaging device positioned proximate to a bed and oriented to capture images of the subject lying in the bed. The system also includes a processing unit in communication with the imaging device and operative to receive the captured images of the subject lying in the bed, the captured images including a plurality of image frames, the processing unit comprising one or more processors and memory. The processing unit includes a pose estimation module trained with a dataset of lying poses and operative to estimate poses of the subject lying in the bed based on one or more of the image frames. The processing unit also includes a posture classification module trained with the dataset of lying poses and operative to classify positions of the subject lying in the bed based on one or more of the image frames. The processing unit is operative to determine a pose and posture of the subject lying in the bed. The method for in-bed pose and posture determination and tracking also includes receiving, by the processing unit of the system, captured images of a human subject lying in a bed from the imaging device of the system in communication with the processing unit. The method for in-bed pose and posture determination and tracking also includes estimating, by the pose estimation module of the processing unit, poses of the subject lying in the bed based on one or more of the image frames. The method for in-bed pose and posture determination and tracking also includes classifying, by the posture classification module of the processing unit, positions of the subject lying in the bed based on one or more of the image frames. The method for in-bed pose and posture determination and tracking also includes determining, by the processing unit, the pose and posture of the subject lying in the bed.

In some embodiments, the method for in-bed pose and posture determination and tracking also includes capturing, by the imaging device, the captured images of the human subject lying in the bed through bedding covering the subject. In some embodiments, the pose estimation module includes a stacked hourglass model trained with the dataset of lying poses. In some embodiments, the posture estimation model includes an autoencoder. In some embodiments, the autoencoder is a histogram of oriented gradients (HoG)-autoencoder. In some embodiments, the method for in-bed pose and posture determination and tracking also includes computing, by a preprocessor of the processing unit, HoG features of each of the one or more images received by the processing unit to form a HoG feature vector corresponding to each respective one of the one or more images. In some embodiments, the method for in-bed pose and posture determination and tracking also includes receiving, by an encoder of the HoG-autoencoder, at least one of the HoG feature vectors formed by the preprocessor. In some embodiments, the method for in-bed pose and posture determination and tracking also includes converting, by the encoder of the HoG-autoencoder, each respective HoG feature vector to a latent vector comprising a low-dimensional representation of the corresponding HoG feature vector. In some embodiments, the method for in-bed pose and posture determination and tracking also includes remapping, by a decoder of an output layer of the HoG-autoencoder, the latent vector to a HoG feature vector. In some embodiments, the method for in-bed pose and posture determination and tracking also includes determining, by a linear classification layer of the HoG-autoencoder, a posture class probability for the corresponding image. In some embodiments, the method for in-bed pose and posture determination and tracking also includes classifying, by a single linear layer of the posture estimation model, positions of the person lying in the bed based on pose estimation model keypoints generated by the pose estimation module.

In some embodiments, the method for in-bed pose and posture determination and tracking also includes confirming, by a motion detection module of the processing unit, a stable state of the subject lying in the bed if a same posture is returned from the posture classification module after a predetermined number of consecutive image frames. In some embodiments, the method for in-bed pose and posture determination and tracking also includes receiving, at an edge device in communication with the processing unit, images from the processing unit. In some embodiments, the method for in-bed pose and posture determination and tracking also includes requesting, at the edge device, notification of a detection or a duration of detection of at least one of a type of pose or a type of posture classification by the processing unit. In some embodiments, the method for in-bed pose and posture determination and tracking also includes initiating, at the edge device, an alarm responsive to the notification of the detection or duration of detection of the at least one of the type of pose or the type of posture classification corresponding to one or more of a proscribed pose, a proscribed posture classification, or an exceeded duration of a proscribed pose or proscribed posture corresponding to one or more alarm limits. In some embodiments, the alarm limits are configurable according to one or more needs, conditions, or goals of the subject. In some embodiments, the needs, conditions, or goals of the subject include at least one of prevention or treatment of pressure ulcers, avoiding supine posture, 3^{rd} trimester pregnancy, sleep apnea, chronic respiratory problems, post-surgical monitoring/recovery, neck or back injury, carpel tunnel syndrome, sleep disorders, fibromyalgia syndrome.

In another aspect, a method to aid in diagnosing, treating, or preventing a sleep-related medical condition is provided. The method to aid in diagnosing, treating, or preventing a sleep-related medical condition includes providing a system for in-bed pose and posture determination and tracking for a human subject. The system includes an imaging device comprising one or more of a depth sensor or a long wavelength infrared camera, the imaging device positioned proximate to a bed and oriented to capture images of the subject lying in the bed. The system also includes a processing unit in communication with the imaging device and operative to receive the captured images of the subject lying in the bed, the captured images including a plurality of image frames, the processing unit comprising one or more processors and memory. The processing unit includes a pose estimation module trained with a dataset of lying poses and operative to estimate poses of the subject lying in the bed based on one or more of the image frames. The processing unit also includes a posture classification module trained with the dataset of lying poses and operative to classify positions of the subject lying in the bed based on one or more of the image frames. The processing unit is operative to determine a pose and posture of the subject lying in the bed. The method to aid in diagnosing, treating, or preventing a sleep-related medical condition also includes acquiring images of the subject using the system while the subject is sleeping or attempting to sleep in a bed for a period of time. The method to aid in diagnosing, treating, or preventing a sleep-related medical condition also includes performing a method for in-bed pose and posture determination and tracking using the acquired images, thereby determining pose and posture of the subject during the period of time or a portion thereof. The method for in-bed pose and posture determination and tracking includes receiving, by the processing unit of the system, captured images of a human subject lying in a bed from the imaging device of the system in communication with the processing unit. The method for in-bed pose and posture determination and tracking also includes estimating, by the pose estimation module of the processing unit, poses of the subject lying in the bed based on one or more of the image frames. The method for in-bed pose and posture determination and tracking also includes classifying, by the posture classification module of the processing unit, positions of the subject lying in the bed based on one or more of the image frames. The method for in-bed pose and posture determination and tracking also includes determining, by the processing unit, the pose and posture of the subject lying in the bed. The method to aid in diagnosing, treating, or preventing a sleep-related medical condition also includes analyzing the pose and/or posture determinations to aid in diagnosing, treating, or preventing the sleep-related medical condition.

In some embodiments, the medical condition is selected from the group consisting of pressure ulcers, avoiding supine posture, 3^{rd} trimester pregnancy, sleep apnea, chronic respiratory problems, post-surgical monitoring/recovery, neck or back injury, carpel tunnel syndrome, sleep disorders, and fibromyalgia syndrome.

Additional features and aspects of the technology include the following:

1. A system for in-bed pose and posture determination and tracking for a human subject, comprising:
an imaging device comprising one or more of a depth sensor or a long wavelength infrared camera, the imaging device positioned proximate to a bed and oriented to capture images of the subject lying in the bed; and
a processing unit in communication with the imaging device and operative to receive the captured images of the subject lying in the bed, the captured images including a plurality of image frames, the processing unit comprising one or more processors and memory, the processing unit including:
   a pose estimation module trained with a dataset of lying poses and operative to estimate poses of the subject lying in the bed based on one or more of the image frames, and
   a posture classification module trained with the dataset of lying poses and operative to classify positions of the subject lying in the bed based on one or more of the image frames;
wherein the processing unit is operative to determine a pose and posture of the subject lying in the bed.
   2. The system of feature 1, wherein the imaging device is capable of imaging body pose and posture of the subject through bedding covering the subject.
   3. The system of any of features 1-2, wherein the processing unit is integrated with the imaging device.
   4. The system of any of features 1-3, wherein the processing unit is located remotely from the imaging device and in electronic communication with the imaging device.
   5. The system of feature 4, wherein the processing unit is located in a cloud-based server.
   6. The system of any of features 1-5, wherein the pose estimation module includes a stacked hourglass model trained with the dataset of lying poses.
   7. The system of any of features 1-6, wherein the posture classification module includes an autoencoder.
   8. The system of feature 7, wherein the autoencoder is a histogram of oriented gradients (HoG)-autoencoder.
   9. The system of feature 8, wherein the processing unit further comprises a preprocessor configured to compute HoG features of each of the one or more images received by the processing unit to form a HoG feature vector corresponding to each respective one of the one or more images.
   10. The system of feature 9, wherein the HoG-autoencoder includes an encoder configured to: receive at least one of the HoG feature vectors formed by the preprocessor; and convert each respective HoG feature vector to a latent vector comprising a low-dimensional representation of the corresponding HoG feature vector.
   11. The system of feature 10, wherein the HoG-autoencoder further comprises:
an output layer including a decoder trained to remap the latent vector to a HoG feature vector; and
a linear classification layer configured to determine a posture class probability for the corresponding image.
   12. The system of any of features 1-11, further comprising an edge device in communication with the processing unit and operative to:
receive images from the processing unit; and
request notification of a detection of a type or duration of pose or posture by the processing unit.
   13. The system of feature 12, wherein the imaging device and the processing unit are integrated within the edge device.
   14. The system of any of features 1-13, further comprising a motion detection module operative to determine if a same posture is returned from the posture classification module after a predetermined number of consecutive image frames.
   15. The system of any of features 1-14, wherein the posture estimation model includes a single linear layer operative to classify positions of the subject lying in the bed based on pose estimation keypoints generated by the pose estimation module.
   16. A method for in-bed pose and posture determination and tracking, comprising:
providing the system of claim 1;
receiving, by the processing unit of the system, captured images of a human subject lying in a bed from the imaging device of the system in communication with the processing unit;
estimating, by the pose estimation module of the processing unit, poses of the subject lying in the bed based on one or more of the image frames;
classifying, by the posture classification module of the processing unit, positions of the subject lying in the bed based on one or more of the image frames; and
determining, by the processing unit, the pose and posture of the subject lying in the bed.
   17. The method of feature 16, further comprising capturing, by the imaging device, the captured images of the human subject lying in the bed through bedding covering the subject.
   18. The method of any of features 16-17, wherein the pose estimation module includes a stacked hourglass model trained with the dataset of lying poses.
   19. The method of any of features 16-18, wherein the posture estimation model includes an autoencoder.
   20. The method of feature 19, wherein the autoencoder is a histogram of oriented gradients (HoG)-autoencoder.
   21. The method of feature 20, further comprising computing, by a preprocessor of the processing unit, HoG features of each of the one or more images received by the processing unit to form a HoG feature vector corresponding to each respective one of the one or more images.
   22. The method of feature 21, further comprising:
receiving, by an encoder of the HoG-autoencoder, at least one of the HoG feature vectors formed by the preprocessor; and
converting, by the encoder of the HoG-autoencoder, each respective HoG feature vector to a latent vector comprising a low-dimensional representation of the corresponding HoG feature vector.
   23 The method of feature 22, further comprising:
remapping, by a decoder of an output layer of the HoG-autoencoder, the latent vector to a HoG feature vector; and
determining, by a linear classification layer of the HoG-autoencoder, a posture class probability for the corresponding image.
   24. The method of any of features 16-23, further comprising classifying, by a single linear layer of the posture estimation model, positions of the person lying in the bed based on pose estimation model keypoints generated by the pose estimation module.
   25. The method of any of features 16-24, further comprising confirming, by a motion detection module of the processing unit, a stable state of the subject lying in the bed if a same posture is returned from the posture classification module after a predetermined number of consecutive image frames.
   26. The method of any of features 16-25, further comprising:
receiving, at an edge device in communication with the processing unit, images from the processing unit; and
requesting, at the edge device, notification of a detection or a duration of detection of at least one of a type of pose or a type of posture classification by the processing unit.
   27. The method of feature 26, further comprising initiating, at the edge device, an alarm responsive to the notification of the detection or duration of detection of the at least one of the type of pose or the type of posture classification corresponding to one or more of a proscribed pose, a proscribed posture classification, or an exceeded duration of a proscribed pose or proscribed posture corresponding to one or more alarm limits.
   28 The method of feature 27, wherein the alarm limits are configurable according to one or more needs, conditions, or goals of the subject.
   29. The method of feature 28, wherein the needs, conditions, or goals of the subject include at least one of prevention or treatment of pressure ulcers, avoiding supine posture, 3^{rd} trimester pregnancy, sleep apnea, chronic respiratory problems, post-surgical monitoring/recovery, neck or back injury, carpel tunnel syndrome, sleep disorders, fibromyalgia syndrome.
   30. A method to aid in diagnosing, treating, or preventing a sleep-related medical condition, the method comprising
providing the system of any of features 1-15;
acquiring images of the subject using the system while the subject is sleeping or attempting to sleep in a bed for a period of time;
performing the method of any of features 16-29 using the acquired images, thereby determining pose and posture of the subject during the period of time or a portion thereof; and
analyzing the pose and/or posture determinations to aid in diagnosing, treating, or preventing the sleep-related medical condition.
   31. The method of feature 30, wherein the medical condition is selected from the group consisting of pressure ulcers, avoiding supine posture, 3^{rd} trimester pregnancy, sleep apnea, chronic respiratory problems, post-surgical monitoring/recovery, neck or back injury, carpel tunnel syndrome, sleep disorders, and fibromyalgia syndrome.

### DESCRIPTION OF THE DRAWINGS

Embodiments of the present disclosure are described by the way of example with references to the accompanying drawings, which are schematic and are not intended to be drawn to scale. The drawings referenced herein form a part of the specification. Features shown in the drawings are meant as illustrative of only some aspects of the presently disclosed embodiments.

FIG. 1A illustrates a prototype of an embodiment of an AI-driven in-bed pose and posture tracking system in a living room.

FIG. 1B illustrates samples of RGB (left column), depth (middle column), and LWIR (right row) of pose/posture images for SLP dataset in different cover conditions.

FIG. 1C illustrates an embodiment including a server-client configuration (e.g., using a cloud-based server). The server runs the inference continuously, while the client is on a mobile platform, which gives the user both live-steam access as well as behavioral tag notifications.

FIG. 1D illustrates data flow of an edge pose and posture tracking system. The infrared (IR) imaging module connects to the edge device (an Android phone here) and transmits the frames to the mobile processing flow. The posture classification results are continuously passed to the motion detection and alarm mode trackers if they are active. When these modes are triggered via their respective conditions, they then run the pose classification model on the current frame and display the results to the user.

FIGS. 2A-2B illustrate a comparison between two sample LWIR images, wherein FIG. 2A is from the SLP dataset, and FIG. 2B was collected using a FLIR One camera. The lower sensor resolution of the FLIR camera module creates a patchier image representing larger areas with the same temperature, losing the finer details captured in the SLP dataset.

FIG. 2C illustrates a screenshot of the Android app performing inference on a given input LWIR frame from the SLP dataset.

FIGS. 3A and 3B illustrate a configurable posture tracking alarm system. To activate the alarm mode the user can toggle an “Alarm Mode” switch. The user can then select the posture that they want to be alerted for from the drop-down menu below (e.g., Left Lying as in FIG. 3A or Supine as in FIG. 3B). The selected posture classification model can keep running on every frame in the background, and upon finding a stable set of frames that depict the posture that is set as an alert, the user receives an alert.

FIG. 4A shows a PCK plot for a pose estimation model tested on the validation set of the SLP dataset.

FIG. 4B shows a confusion matrix for a HoG-autoencoder posture classification model running on the SLP dataset, representing the distribution of predicted classes versus their ground truth values. The model appears to have a harder time distinguishing between left and right lying postures than between the supine position and the other two.

FIGS. 4C-4D illustrate a comparison of confusion matrices for two different posture classification models running on a real-life dataset collected via the system, wherein FIG. 4C used a pose-based model and FIG. 4D used a HOG-autoencoder model.

### DETAILED DESCRIPTION

### 1. AI-Implemented In-Bed Pose and Posture Tracking System

Every person spends around ⅓ of their life in bed. For an infant (i.e., human subjects 0 to 1 years of age) or a young toddler (i.e., human subjects 1 to 2 years of age), this percentage can be much higher. Additionally, for bed-bound patients of any age, including, for example, infants, young toddlers, juveniles (i.e., human subjects of 1 to 18 years of age), or adults (i.e., human subjects over 18 years of age), it can go up to 100% of their time. In-bed pose/posture estimation is a critical step in any human behavioral monitoring systems, which are focused on prevention, prediction, and management of at-rest or sleep-related conditions in both adults and children. Pose-a collection of human joint locations—is a succinct representation of a person's physical state, and postures are defined as a disposition of a few particular body parts with respect to each other and a locomotion surface (e.g., supine, left side, etc.). Automatic non-contact human pose/posture estimation topics have received a lot of attention/success especially in the last few years in the artificial intelligence (AI) community thanks to the introduction of deep learning and its power in AI modeling. However, the state-of-the-art vision-based AI algorithms in this field are unreliable at best and typically do not work under the challenges associated with in-bed human behavior monitoring, which includes significant illumination changes (e.g., full darkness at night), heavy occlusion (e.g., covered by sheets, blankets, quilts, and/or other bedding/bedcoverings), as well as privacy concerns that mitigate large-scale data collection, necessary for any AI model training. The data quality challenges and privacy concerns have hindered the use of AI-based in-bed pose and posture tracking system at home, where there is an unmet need for an unobtrusive and privacy-preserving monitoring technology that can be installed and used in the comfort of one's bedroom. The impact of such technology is magnified when the person being monitored is an expectant mother who needs to avoid sleeping on her back in the last trimester for her comfort and the safety of the fetus, or an infant who needs to be kept on her back to reduce the risk of sudden infant death syndrome (SIDS), among other emerging applications of sleep monitoring.

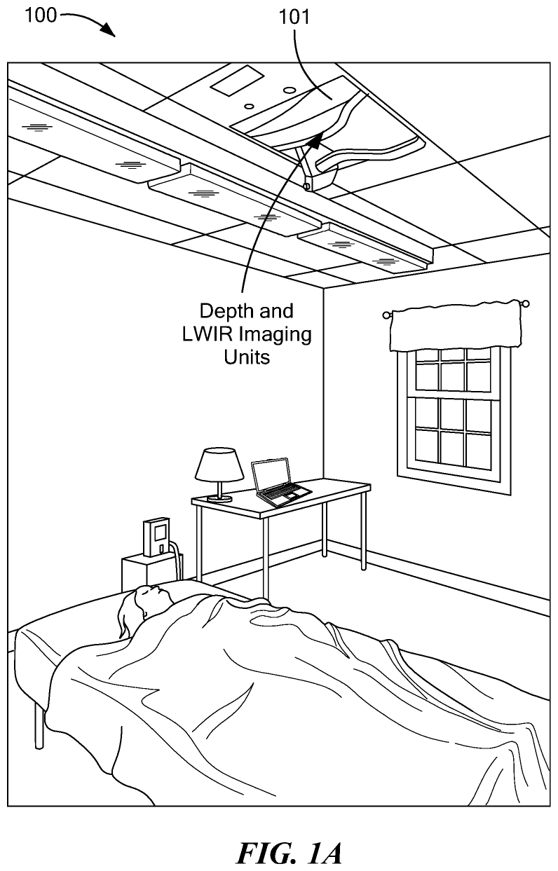

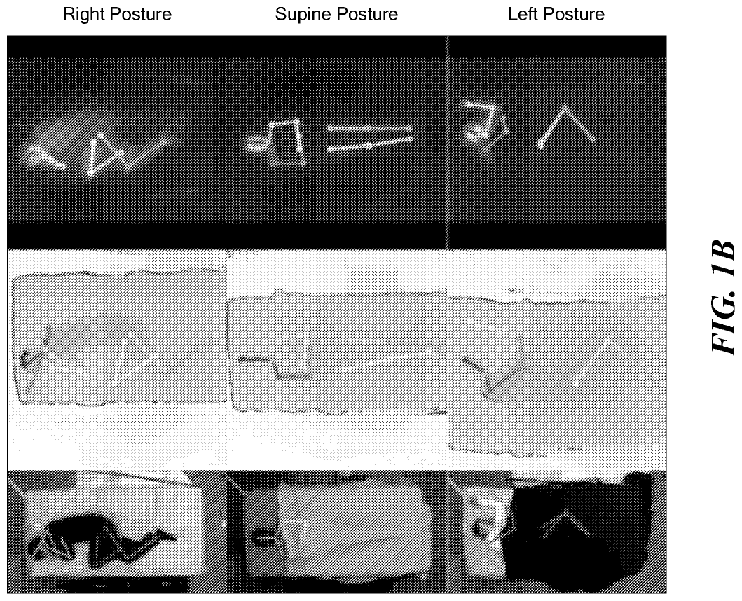

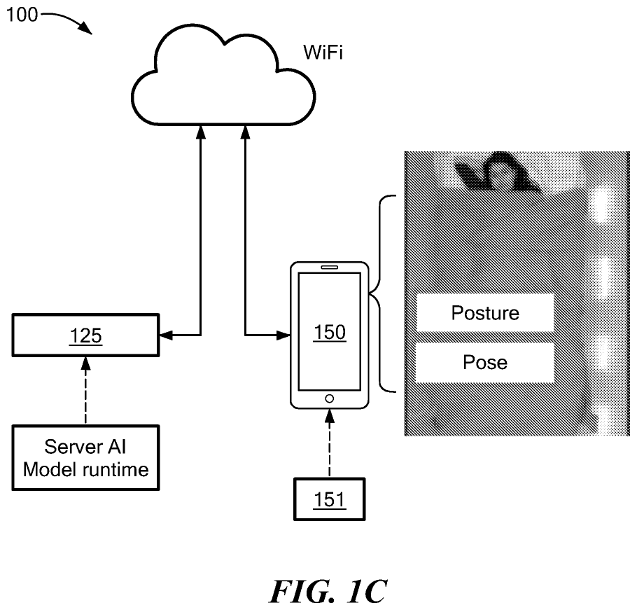

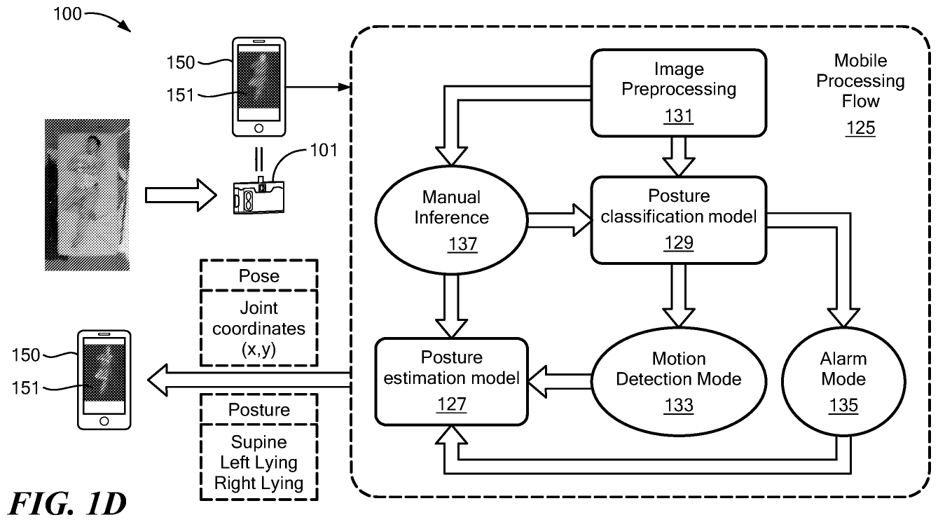

The technology described herein provides a non-contact, low-cost, and privacy-preserving solution as a long-term and robust AI-driven in-bed pose and posture tracking system, which can be fully functional under these challenging conditions. An initial prototype of an AI-driven in-bed pose and posture tracking system 100 setup in a living room is shown in FIG. 1A. The AI modeling parts of the in-bed pose and posture tracking system can enable implementation of a stand-alone, non-RGB monitoring system. Embodiments of the in-bed pose and posture tracking system 100 can provide affordable solutions for sleep or in-bed behavior tracking purposes, including, but not limited to: (1) sleep posture monitoring for pregnant women; (2) infant pose/posture monitoring for daily checkup as well as early developmental behavior studies; and (3) continuous bed-bound patient monitoring in locations such as nursing homes and hospitals.

As shown in FIGS. 1A & 1D, the in-bed pose and posture tracking system 100 includes a non-RGB camera 101 mounted over a bed/crib and an embedded processing unit 125 that directly estimates the subject's pose and posture with high temporal resolution via an already-trained AI model. The two non-RGB imaging modalities that have been tested in initial prototypes are depth and long wavelength IR (LWIR), both of which are capable of providing the visual clues even under full darkness and heavy occlusion. The sensing module of the in-bed pose and posture tracking system 100 can be one of the many commercial off-the-shelf depth or LWIR cameras, including the Intel RealSense RGBD camera or the FLIR thermal camera. The implementation of the in-bed pose and posture tracking system 100 then can be depth based, LWIR based, or a combination thereof (see depth and LWIR image examples in FIG. 1B). Some advantages of the in-bed pose and posture tracking system 100 are achieved by use of: (1) a large-scale multimodal dataset called SLP specifically collected and carefully annotated for in-bed human pose and posture, and (2) a series of pose and posture estimation models with deep neural network architectures trained using SLP datasets, which robustly work on different candidate imaging modalities in different settings (see FIG. 1B). The technology also includes a privacy-preserving in-bed pose and posture tracking system 100 running entirely on an edge device with added functionality to detect stable motion as well as setting user-specific alerts for given poses. The estimation accuracy of the system has been evaluated on a series of retrospective infrared (LWIR) images as well as samples from a real-world test environment. The test results reached over 93.6% estimation accuracy for in-bed poses and achieved over 95.9% accuracy in estimating three in-bed posture categories.

The system 100, in some embodiments, can be fully integrated into a single device (e.g., a mobile edge device as shown in FIG. 1D) with inclusion of a camera 101, an embedded processing unit 125, and an app 151/device 150 for streaming/delivering the pose and posture data. FIG. 1C illustrates a workflow of the in-bed pose and posture tracking system 100 implemented in a server-client configuration wherein the processing unit 125 and related functionalities are implemented in a separate server whether a local physical serve or a cloud-based server. The in-bed pose and posture tracking system 100 can capture the in-bed human image and estimate the pose and posture at endpoint. The app 151 on the user's smart phone 150 communicates with the processing unit 125 (e.g., the local/cloud server as in FIG. 1C or the integrated processing unit 125 of FIG. 1D) via the Internet, with functions including: (1) live-stream of the pose and posture of the in-bed user; (2) record and display of the pose and posture history over a selected period of time; and (3) optional notification set by the user if a specific pose/posture has been registered such as “Supine Posture Avoidance” for pregnant women. After installing the device over the bed to have a bird's eye view, the user can be instructed to install an in-bed pose and posture tracking system app and to select a WiFi account to connect. After this setup and each reboot, the device can automatically start the server by (1) running the pose and posture inference, and (2) listening to the client request. In no live stream mode, the system can run with low frequency and the frame rate can only be increased when large variations occur in the scene.

### 2. Implementation of In-Bed Pose and Posture Tracking System

Referring to FIG. 1D, an embodiment of a working pipeline of the system 100 is illustrated with major components including image preprocessing 131, posture classification 129, pose estimation, 127, motion detection 133, and customizable alarm notification modules 135. The system can be trained using LWIR pose images from the Simultaneously collected multimodal Lying-Pose (SLP) dataset. Aiming at a portable edge computing tool 150, the system 100 can be compatible with smart phone devices 150, such as an Android-based device, which enables attachment of an off-the-shelf thermal imaging camera 101.

### 2.1. Hardware Configuration

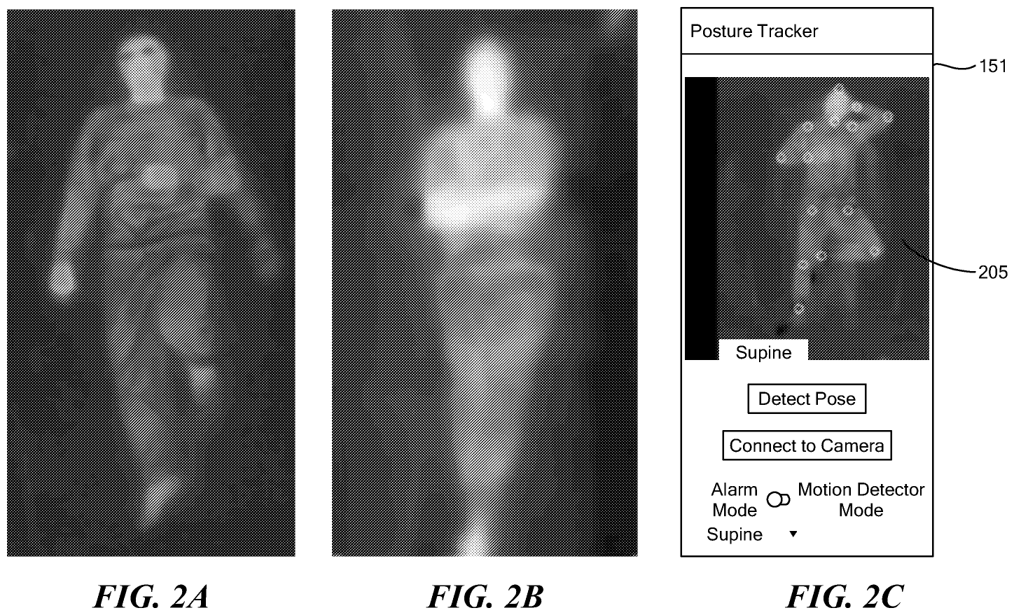

To preserve a person's privacy while in bed, LWIR can be used as a sensing modality by using the FLIR One thermal camera module which is compatible with many Android and iOS powered phones. The camera module has the speed of 8.7 Hz and is equipped with a thermal sensor with the resolution of 60×80. It has a temperature range of −20° C. to 120° C. with an accuracy of +3° C. and thermal sensitivity of 150 m° C. Two samples of LWIR images from the SLP dataset and captured from the FLIR One camera are shown in FIGS. 2A & 2B.

### 2.2. Inference Modules

Existing computer vision approaches are modified for depth or LWIR and used as backstage inference models including the stacked hourglass model for pose estimation and a lightweight histogram of oriented gradients (HoGs) feature based linear model for real-time posture classification.

### 2.2.1. In-Bed Pose Estimation Via Pose Estimation Module

For pose estimation in the pose estimation module 127, hourglass networks are designed to capture information at multiple scales and bring the features from these scales together to output pixel-wise predictions. The network has a single pipeline with skip layers to preserve spatial information at each resolution. The output of the network is a set of heatmaps indicating the probability of a joint's presence at each pixel. While the network was originally designed to work with 3-channel RGB images, modifying for use with single channel LWIR images is a viable use case. Existing network setups are used to train a stacked hourglass network to extract the coordinates from a human body using only LWIR image as input.

### 2.2.2. In-Bed Posture Classification Via Posture Classification Module

For posture classification in the posture classification module 129, at least one of two different approaches is implemented: (1) an autoencoder that learns the mapping between input feature vectors from the LWIR image to a latent space for posture classification, or (2) a single linear layer that takes the keypoint coordinates from the pose estimation model directly for posture classification.

HoG-autoencoder posture classification: This model takes in the HoG features of the image as the input vector, which is fed to an encoder including a single hidden layer that converts the HoG features to a latent vector that is a low dimensional representation (i.e., the outcome of a dimension reduction process on high-dimensional data) of the original vector. The output of this encoder is fed to two separate output layers, (1) a decoder that learns to remap the latent vector back to a HoG feature vector, and (2) a linear classification layer that outputs the posture class probability.

Pose-based posture classification: This approach involves taking in the output of the pose estimation model (keypoints) and training a single linear layer that outputs the posture class probability. While this measure is shown to have higher precision, its accuracy is dependent on the accuracy of the pose estimation model, while also consequently being slower. In one embodiment, three distinct types of postures are used, supine, left lying, and right lying, as the output labels for the classification. While postures can also be reliably classified by using the output coordinates from the pose estimation model, to make posture estimation run much faster on an edge device, and to be agnostic to any incoming pose estimation error, the HoG-autoencoder based approach can be implemented for posture classification in the Android framework.

### 2.2.3. Motion Detection Via Motion Detection Module

The motion detection module 133 can be implemented to wait an interval to reassess posture to determine whether the subject is in a stable pose. For example, in some embodiments, the motion detection module 133 can be configured such that it waits for 50 consecutive frames (roughly 2-3 seconds) and if the same posture is returned from the classification model it determines that the subject is in a stable pose. Upon confirming that this stable state has been achieved, the system 100 then automatically runs the pose estimation model on the next frame to get the joint coordinates of the subject. This is much more efficient as the posture classification model only has a single hidden layer, and only requires computing the HoG features for a frame, and thus takes a much shorter time per image.

### 2.3. Android Framework

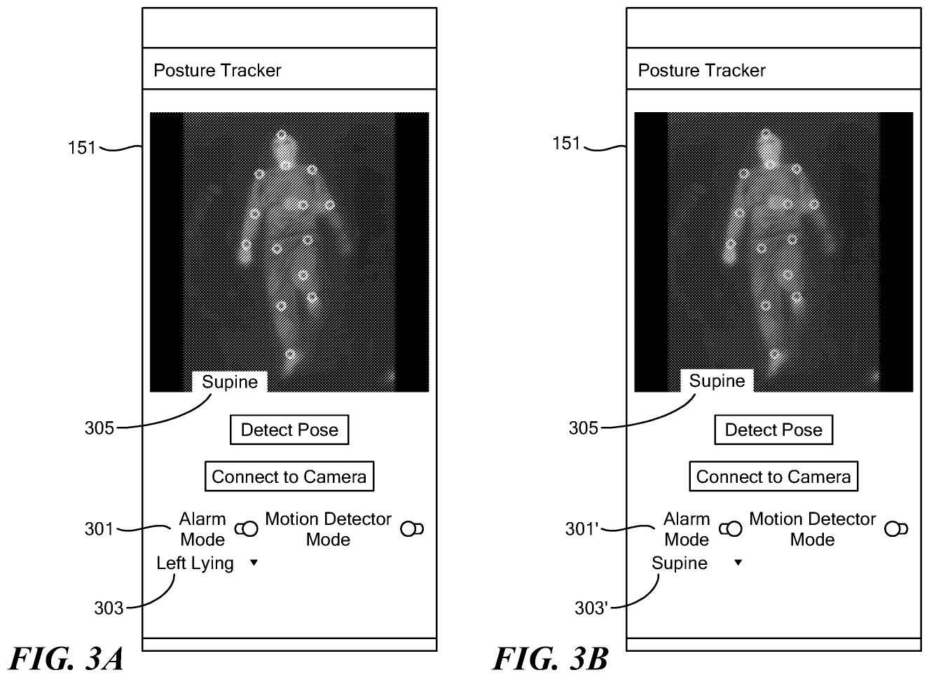

In the technology described herein, the Android system is employed as the platform for edge computing which can be run on most of the portable devices 150 such as a cell phone. The app 151 provides a few fundamental user interface (UI) components, as illustrated in FIG. 2C and FIGS. 3A-3B, and is optimized to perform various operations in real-time including (1) Detect Pose button, (2) Connect to Camera button, (3) Motion Detection switch, (4) Alarm Mode Switch, and (5) Alarm Posture selection. Some key technique implementation includes:

Interface with FLIR One Camera: The FLIR One SDK can be used to create the interface between the Android app and the FLIR One camera. A USB port listener is setup on start of the app, and once the “Connect to Camera” button is pressed, a connection is established to the camera module. This creates a stream of LWIR images, which pass through some basic preprocessing steps.

Model Deployment and Optimization: Pose estimation models are conventionally implemented with PyTorch. For Android deployment, the model is first translated into high performance Torchscript, which is platform independent. Specific optimizations can be conducted during this process including fusing common layers and dropout removal. This optimized model can then directly be loaded into the Android App.

LWIR Image Preprocessing via Preprocessing Module: In the preprocessing module 131 First, a perspective transform is performed on the LWIR images to ensure that the image view is parallel to the plane of the camera. The sides of the frame are then padded to get a square frame to pass on to the pose and posture models. The HoG features of each frame are also computed for use in the posture classification model. This calculation is performed for each frame since it is used in the motion detection mode as well.

Manual-Triggered Inference via Manual Inference Module: In some embodiments, manual inference module 137 can include a button titled “Detect Pose” is provided to manually chose running the pose and posture models at any given time. This process takes the HoG features and the current frame as the input and runs them through the pose estimation and posture classification models. The coordinates and labels are extracted from the output, and confidence probabilities of the results are used to determine whether the given pose is accurate within a certain threshold. The results are then drawn on the current frame and the annotated result 205 is displayed on the screen 151 as FIG. 2C shows.

Alarm Mode via Mobile App: The alarm module 135 functionality can be further extended by allowing the user to set alarms 301 for certain postures, as seen in FIGS. 3A-3B. In the screen 151 of FIG. 3A, it can be seen that enabling an alarm 301 for left lying postures 303 does not throw up an alarm, while on the screen 151 of FIG. 3B, it can be seen that setting an alarm 301′ for the supine position 303′ alerts the user when a supine posture is detected 305.

### 3. Experimental Evaluation

### 3.1. Evaluation:

The system was evaluated on a multimodal in-bed pose dataset, called SLP as well as in a real working environment. In the real-life test, the posture estimation pipeline can reach 25 fps and pose estimation for 0.85 fps.

### 3.1.1. Evaluation Using SLP Dataset

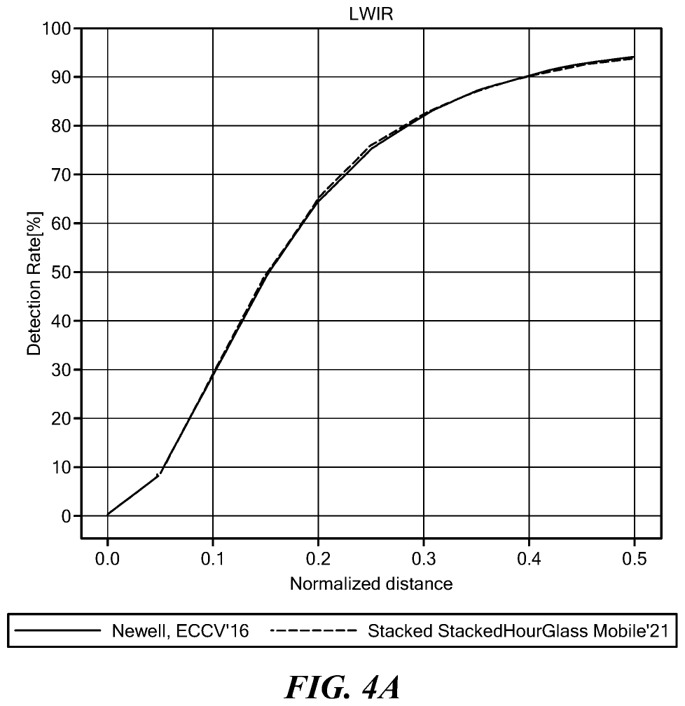

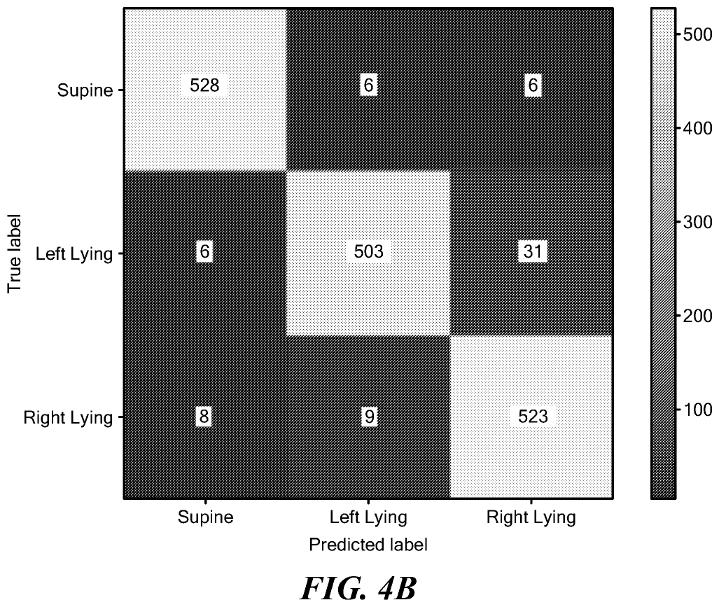

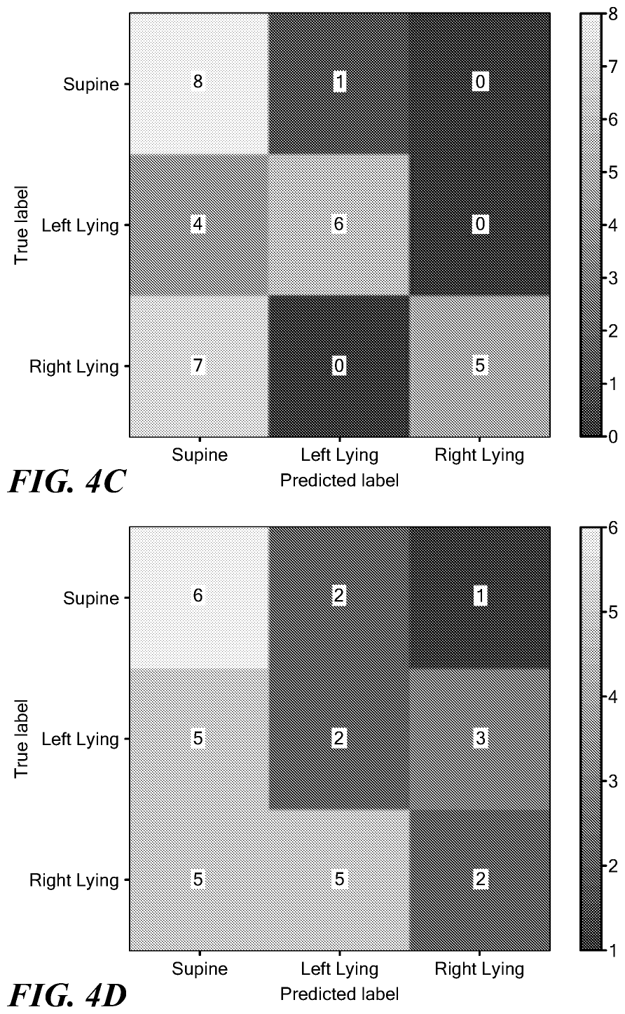

Both pose estimation and posture classification networks have been trained and evaluated on the SLP dataset. This dataset has 102 subjects, each with 45 images for every cover type (i.e., uncovered, thin cover, and thick cover) for a total of 13,770 annotated images. Out of these subjects, 90 were used for the training of the models and 12 were kept for the validation purpose. The confusion matrix for the HoG-autoencoder posture model is given in FIG. 4B. The percentage of correct keypoint (PCK) values for pose estimation was also compared between an existing, heavier-weight pose estimation model and the present on-edge mobile model in FIG. 4A and found them to be very similar and comparable. The results show that the scripted mobile model is in line with the original implementation, and the optimizations performed by Torchscript did not reduce the performance significantly. The PCK results for a threshold of 0.5 are shown in Table 1. The final accuracy values on the validation set for pose and posture estimation are 93.6 (PCKh@0.5) and 95.9, respectively.

TABLE 1
Pose estimation accuracy (per body part) of applying the edge
systems described herein to the validation set of the SLP dataset.

| | Body Parts | | | | |
|---|---|---|---|---|---|
| | R-Ankle | R-Knee | R-Hip | L-Hip | L-Knee |
| PCK@0.5(%) | 95.7 | 96.5 | 85.1 | 86.5 | 96.4 |
| | Body Parts | | | | |
| | L-Ankle | R-Wrist | R-Elbow | R-Shoulder | L-Shoulder |
| PCK@0.5(%) | 96.9 | 89.9 | 93.8 | 92.8 | 94.8 |
| | Body Parts | | | | |
| | L-Elbow | L-Wrist | Thorax | Head | Total |
| PCK@0.5(%) | 94.8 | 90.3 | 99.3 | 98.1 | 93.6 |

### 3.1.2. Evaluation on Real-Life Data

For real field test, a small dataset was collected with the mobile device for posture classification.

Testing the accuracy of the models in real life conditions proved to be more challenging, especially since the LWIR cameras designed for the edge devices are not comparable to the high-quality cameras used in capturing data in the SLP dataset. Thus, the sensor resolution is a lot lower and the corresponding heatmap is more pixelated as a result (see FIG. 2B). The pose estimation model fared much better in this regard, while the single layer posture classification model was unable to capture information at the same scale and performed worse on images from the low-resolution sensors. A small test sample was collected to show the differences between the HoG-autoencoder and the pose-based posture classification models. On comparing the results between the two, FIGS. 4C and 4D shows that the pose-based based model does marginally better than the HoG-autoencoder model. However, it comes at the cost of longer inference time since it is now dependent on the pose estimation model. The HoG-autoencoder posture model takes an average of 0.04 s to run on a single frame, whereas the pose estimation model takes an average of 1.18 s per frame, which is orders of magnitude slower when used in pose-based posture classification.

### 4. Advantages of the Technology

Compared to the existing AI approaches for human in-bed behavior monitoring, which are mainly based on body-worn inertial measurement units (IMUs) or pressure mat (PM) sensing, the in-bed pose and posture tracking system described herein is (1) fully non-contact, so unobtrusive; (2) low-cost, so affordable for many customers; (3) has smaller form factor, so can be installed easily and safely in many bed settings; (4) easy to installed and maintained, so can be setup by non-technical users; (5) safe, with no radiation or infection harm, and does not need frequent cleaning; (6) privacy-preserving, due to being non-RGB as well its in-situ processing; and (7) provides higher granularity in behavior recognition ability, since it is based on body pose and posture over time. Taking depth modality for example, the initial depth based in-bed pose and posture tracking system prototype evaluation using SLP dataset and tested on more than 100 in-bed participants already achieved an accuracy as high as 94.2% for pose estimation and 99.8% for posture detection in a home setting scenario, and an accuracy of 94.1% and 92.5% for pose and posture estimation in a fully novel hospital setting, respectively. Compared to the existing pressure map (PM) solution designed for in-bed posture tracking, the in-bed pose and posture tracking system is only 1/60 in cost and 1/300 in size, but with much higher posture detection accuracy as well as higher body pose granularity. Cameras for use with the technology are prevalent and can be easily maintained. The technology is fully non-contact and unobtrusive and is AI-driven and smart.

Due to these features and advantages, the in-bed pose and posture tracking system can provide a complete pipeline ready for real-world applications. In some applications, the system can be employed for in-bed pose/posture monitoring in the last trimester of pregnancy and re-purposed as a baby monitor afterwards.

Embodiments of the in-bed pose and posture tracking system can bring affordable solutions to many customers, whether private individuals or healthcare providers, for sleep or in-bed behavior tracking purposes. In this regard, the technology can be used in connection with a variety of applications including, for example, for or with baby monitors; for sleep posture monitoring for pregnant women and families, by support groups for pregnant mothers, including Doulas, yoga for pregnancy, gift registries, healthcare providers including sleep specialists and OBGYN, physical therapists, bedridden nursing services or at-home care for bedridden or partially bedridden patients, sleep study centers, monitoring sleep position after critical surgery, pose/posture monitoring for daily checkups as well as developmental behavior studies, continuous bed-bound patient monitoring in nursing homes or hospitals, management and prevention of pressure ulcers, specific posture avoidance (e.g., supine posture avoidance during 3^{rd} trimester pregnancy), sleep apnea, chronic respiratory problems, post-surgical monitoring/recovery, neck or back injury monitoring, carpel tunnel syndrome, sleep disorders, fibromyalgia syndrome, any other relevant conditions, or combinations thereof.

Various advantages and features of the systems and methods described herein include, but are not limited to:

Convenience: Some embodiments can be based on an iOS or Android-friendly portable device with an attached off-the-shelf LWIR camera (e.g., an Android device as shown and described herein).

Versatility: The system implements several primary functions highly needed for home-based in-bed activity monitoring, including active inference, automatic monitoring, and customizable alarm setting.

Privacy Preserving: The long wave IR (LWIR) is used as the main imagining modality, which preserves the privacy of the individual by not capturing any identifying characteristics from them, including their faces, clothing, and the surrounding environments.

Accuracy: The system employs an in-bed human pose estimation model as the backbone model, which reached over 93.6% accuracy on PCKh@0.5 metric for pose estimation. The system also implements a lightweight linear classifier for posture estimation that achieved over 95.9% accuracy in estimating three in-bed posture categories.

As used herein, “consisting essentially of” allows the inclusion of materials or steps that do not materially affect the basic and novel characteristics of the claim. Any recitation herein of the term “comprising,” particularly in a description of components of a composition or in a description of elements of a device, can be exchanged with “consisting essentially of” or “consisting of.”

The present technology has been described in conjunction with certain preferred embodiments and aspects. It is to be understood that the technology is not limited to the exact details of construction, operation, exact materials or embodiments or aspects shown and described, and that various modifications, substitution of equivalents, alterations to the compositions, and other changes to the embodiments and aspects disclosed herein will be apparent to one of skill in the art.

What is claimed is:

1. A system for in-bed pose and posture determination and tracking for a human subject, comprising:
an imaging device comprising one or more of a depth sensor or a long wavelength infrared camera, the imaging device positioned proximate to a bed and oriented to capture images of the subject lying in the bed; and
a processing unit in communication with the imaging device and operative to receive the captured images of the subject lying in the bed, the captured images including a plurality of image frames, the processing unit comprising one or more processors and memory, the processing unit including:
a pose estimation module trained with a dataset of lying poses and operative to estimate poses of the subject lying in the bed based on one or more of the image frames, and
a posture classification module trained with the dataset of lying poses and operative to classify positions of the subject lying in the bed based on one or more of the image frames;
wherein the processing unit is operative to determine a pose and posture of the subject lying in the bed.

2. The system of claim 1, wherein the imaging device is capable of imaging body pose and posture of the subject through bedding covering the subject.

3. The system of claim 1, wherein the processing unit is integrated with the imaging device.

4. The system of claim 1, wherein the processing unit is located remotely from the imaging device and in electronic communication with the imaging device.

5. The system of claim 4, wherein the processing unit is located in a cloud-based server.

6. The system of claim 1, wherein the pose estimation module includes a stacked hourglass model trained with the dataset of lying poses.

7. The system of claim 1, wherein the posture classification module includes an autoencoder.

8. The system of claim 7, wherein the autoencoder is a histogram of oriented gradients (HoG)-autoencoder.

9. The system of claim 8, wherein the processing unit further comprises a preprocessor configured to compute HoG features of each of the one or more images received by the processing unit to form a HoG feature vector corresponding to each respective one of the one or more images.

10. The system of claim 9, wherein the HoG-autoencoder includes an encoder configured to:
receive at least one of the HoG feature vectors formed by the preprocessor; and
convert each respective HoG feature vector to a latent vector comprising a low-dimensional representation of the corresponding HoG feature vector.

11. The system of claim 10, wherein the HoG-autoencoder further comprises:
an output layer including a decoder trained to remap the latent vector to a HoG feature vector; and
a linear classification layer configured to determine a posture class probability for the corresponding image.

12. The system of claim 1, further comprising an edge device in communication with the processing unit and operative to:
receive images from the processing unit; and
request notification of a detection of a type or duration of pose or posture by the processing unit.

13. The system of claim 1, wherein the imaging device and the processing unit are integrated within the edge device.

14. The system of claim 1, further comprising a motion detection module operative to determine if a same posture is returned from the posture classification module after a predetermined number of consecutive image frames.

15. The system of claim 1, wherein the posture estimation model includes a single linear layer operative to classify positions of the subject lying in the bed based on pose estimation keypoints generated by the pose estimation module.

16. A method for in-bed pose and posture determination and tracking, comprising:
providing the system of claim 1;
receiving, by the processing unit of the system, captured images of a human subject lying in a bed from the imaging device of the system in communication with the processing unit;
estimating, by the pose estimation module of the processing unit, poses of the subject lying in the bed based on one or more of the image frames;
classifying, by the posture classification module of the processing unit, positions of the subject lying in the bed based on one or more of the image frames; and
determining, by the processing unit, the pose and posture of the subject lying in the bed.

17. The method of claim 16, further comprising capturing, by the imaging device, the captured images of the human subject lying in the bed through bedding covering the subject.

18. The method of claim 16, wherein the pose estimation module includes a stacked hourglass model trained with the dataset of lying poses.

19. The method of claim 16, wherein the posture estimation model includes an autoencoder.

20. The method of claim 19, wherein the autoencoder is a histogram of oriented gradients (HoG)-autoencoder.

21. The method of claim 20, further comprising computing, by a preprocessor of the processing unit, HoG features of each of the one or more images received by the processing unit to form a HoG feature vector corresponding to each respective one of the one or more images.

22. The method of claim 21, further comprising:
receiving, by an encoder of the HoG-autoencoder, at least one of the HoG feature vectors formed by the preprocessor; and
converting, by the encoder of the HoG-autoencoder, each respective HoG feature vector to a latent vector comprising a low-dimensional representation of the corresponding HoG feature vector.

23. The method of claim 22, further comprising:
remapping, by a decoder of an output layer of the HoG-autoencoder, the latent vector to a HoG feature vector; and
determining, by a linear classification layer of the HoG-autoencoder, a posture class probability for the corresponding image.

24. The method of claim 16, further comprising classifying, by a single linear layer of the posture estimation model, positions of the person lying in the bed based on pose estimation model keypoints generated by the pose estimation module.

25. The method of claim 16, further comprising confirming, by a motion detection module of the processing unit, a stable state of the subject lying in the bed if a same posture is returned from the posture classification module after a predetermined number of consecutive image frames.

26. The method of claim 25, further comprising:
receiving, at an edge device in communication with the processing unit, images from the processing unit; and
requesting, at the edge device, notification of a detection or a duration of detection of at least one of a type of pose or a type of posture classification by the processing unit.

27. The method of claim 26, further comprising initiating, at the edge device, an alarm responsive to the notification of the detection or duration of detection of the at least one of the type of pose or the type of posture classification corresponding to one or more of a proscribed pose, a proscribed posture classification, or an exceeded duration of a proscribed pose or proscribed posture corresponding to one or more alarm limits.

28. The method of claim 27, wherein the alarm limits are configurable according to one or more needs, conditions, or goals of the subject.

29. The method of claim 28, wherein the needs, conditions, or goals of the subject include at least one of prevention or treatment of pressure ulcers, avoiding supine posture, 3^{rd} trimester pregnancy, sleep apnea, chronic respiratory problems, post-surgical monitoring/recovery, neck or back injury, carpel tunnel syndrome, sleep disorders, fibromyalgia syndrome.

30. A method to aid in diagnosing, treating, or preventing a sleep-related medical condition, the method comprising
providing the system of claim 1;
acquiring images of the subject using the system while the subject is sleeping or attempting to sleep in a bed for a period of time;
performing the method of claim 13 using the acquired images, thereby determining pose and posture of the subject during the period of time or a portion thereof; and
analyzing the pose and/or posture determinations to aid in diagnosing, treating, or preventing the sleep-related medical condition.

31. The method of claim 30, wherein the medical condition is selected from the group consisting of pressure ulcers, avoiding supine posture, 3^{rd} trimester pregnancy, sleep apnea, chronic respiratory problems, post-surgical monitoring/recovery, neck or back injury, carpel tunnel syndrome, sleep disorders, and fibromyalgia syndrome.
